(19) United States
(12) Patent Application Publication
(10) Pub. No.: US 2005/0230639 A1
(43) Pub. Date: Oct. 20, 2005

(54) Knife and kitchen tool sterilizer and holder

(75) Inventors: Bruce Ancona, 1 Irving Place, NY (US); Louis Henry, Scarsdale, NY (US)

Correspondence Address: NORMAN H. ZIVIN;Cooper & Dunham LLP, 1185 Avenue of the Americas, New York, NY, 10036, US

(73) Assignee: Kash 'N Gold, Ltd.

(21) Appl. No.: 10/824,666

(22) Filed: Apr. 14, 2004

(51) Int. Cl. A61L 2/10

(52) U.S. Cl. 250/455.11

(57) ABSTRACT

A sterilizer and storage container for kitchen tools, knives and cutting boards which sterilizes such items using ultraviolet light in the UV-C range. The sterilizer features a housing which encloses a receptacle made of a material or structure “transparent” to UV radiation for enclosing items to be sterilized, means for lifting and lowering utensils in said receptacle for ease of filling and emptying said receptacle, a source of UV light, a cover to seal the housing and prevent the leakage of UV light and a starter button to activate the UV light source. When the starter button is activated, UV light is directed through the receptacle and onto the items to be sterilized. The UV light destroys microorganisms on the surfaces of the items to be sterilized. The items are removed from the device in a sterilized state.

### FIELD OF THE INVENTION

The present invention is generally directed to a novel device for sterilizing knives, kitchen tools and cutting boards which can come into contact with, and harbor, harmful microorganisms, including bacteria and viruses. The invention provides a safe, fast and convenient way to disinfect and store such items. The device permits exposure of items placed therein to ultraviolet light, and provides a convenient storage device for the items.

### BACKGROUND OF THE INVENTION

Kitchens and other food preparation areas can have high levels of dangerous microorganisms, including bacteria, which thrive in warm, dark, moist environments. Kitchen items, including knives, tools and cutting boards, which contact meat, soft cheeses and other foods which harbor such microorganisms can spread such germs from one food to another, or from one tool to another. Ultimately, such microorganisms can be ingested in foods and cause serious illness, such as salmonella or e. coli infection. Vigilant cleaning with sufficiently hot water and soap and careful food preparation practices can minimize the risk. However, inconsistent cleaning habits, incorrect water temperature and lax food preparation practices can contribute to the growth of harmful microorganisms in the kitchen. Moreover, simple cleaning in soap and water is sometimes insufficient to kill all microorganisms on a given surface. Traditional cleaning with water also may be ineffective for items which cannot be completely submerged, such as wood, and items which can trap microorganisms in small spaces, such as knife handles. Heat, as from steam or microwaving, is undesirable as it may damage softer plastics, require cool-down time before use, or burn the user. Metal parts such as screws, handles and utensil parts make microwave activated sterilization impossible. Moreover, after such items are cleaned, they may come into contact with contaminated items, such as in a storage bin or drawer. Further, because such items contact food, it is important that they do not maintain any chemical residue from cleaning solutions.

Thus, it is important to provide a system for disinfecting kitchen equipment, such as knives, tools and cutting boards, which can come into contact with, and harbor, harmful microorganisms. Moreover, it is important to insure thorough and complete sterilizing of such items. It also is desirable to provide a storage container for such equipment to insure that after sterilizing, such equipment does not come into contact with other equipment which may harbor dangerous microorganisms.

The present invention addresses the need for a device for disinfecting kitchen equipment, such as knives, tools and cutting boards and provides a compact, attractive counter-top sterilizer which can accommodate a variety of kitchen equipment and which also provides an attractive and convenient storage container for such equipment.

The present invention attains each of these goals through the use of a compact and convenient sterilizer and storage unit which uses ultraviolet, or “UV” light. UV light is invisible radiation having a wavelength of between 100 to 320 nm. The most effective range for sterilization is within 200 nm to 290 nm, designated the UV-C band, with a bandwidth between 250 nm and 260 nm being optimal. At this wavelength, such light is capable of inactivating and destroying a variety of bacteria, viruses, mold and other microorganisms. When UV-C light hits a microorganism, the light is absorbed by the microorganism, which is destroyed. Because the items are “sterilized” by light, they are not wet by, or submerged in, water, soap or chemicals. Thus, no drying or extreme heat is required, and no potentially harmful chemicals or soaps are left behind on the item. Even items that are not suitable to be washed in water, such as wood, can be sterilized with UV light.

### SUMMARY OF THE INVENTION

The present invention is directed to a novel device which comprises a housing, made of material suitable to contain UV radiation, which housing encloses a basket or receptacle made of a material or structure “transparent” to UV radiation, through which UV light may be transmitted for enclosing items to be sterilized, a knife block to receive knives to be sterilized, a source of UV light, reflective material to insure exposure of all surfaces to UV light and a door to seal the housing and prevent the leakage of UV light. When the system is activated, the UV light is directed through the receptacle and into the enclosure onto the items to be sterilized, insuring that UV light hits all surfaces of each piece of equipment to be sterilized. Internal surfaces of the device are coated with reflective material to insure exposure of all surfaces of knives, tools and cutting boards to UV light. The UV light destroys microorganisms on the surfaces of the equipment to be sterilized, but does not remain in or on the equipment.

The equipment is removed from the device in a sterilized state.

### BRIEF DESCRIPTION OF THE DRAWINGS

FIG. 1 shows a front view of an embodiment of the invention.

FIG. 2 shows a front view of the invention opened to show equipment therein.

FIG. 3 shows a front view of the invention opened to show equipment therein.

FIG. 4 shows an exploded view of an embodiment of the invention.

FIG. 5 shows a longitudinal cross-sectional view of the embodiment of FIG. 9 taken approximately along the line A-A of FIG. 9.

FIG. 6 shows a top view of an embodiment of the invention.

FIG. 7 shows a bottom view of an embodiment of the invention.

FIG. 8 shows a side view of an embodiment of the invention.

FIG. 9 shows a rear view of an embodiment of the invention.

### DETAILED DESCRIPTION OF THE PREFERRED EMBODIMENTS

Reference will now be made in detail to the preferred embodiments of the invention, examples of which are also provided in the following description. Exemplary embodiments of this invention are described in some detail, although it will be apparent to those skilled in the relevant art that some features that are not particularly important to an understanding of the invention may not be shown for the sake of clarity.

The present invention is directed to a knife and kitchen tool sterilizer and holder. The housing of the sterilizer is constructed of material that is impervious to UV-C radiation, such as plastic, and contains knife holders for holding knives. The housing is sealed by a door, which provides access to inside the housing. The housing comprises a holder for placing articles to be sterilized, knife holders and a slot for a cutting board, a UV-C light source, a power source, and means for reflecting the UV-C light source such that all surfaces of all items placed therein are exposed to UV-C light.

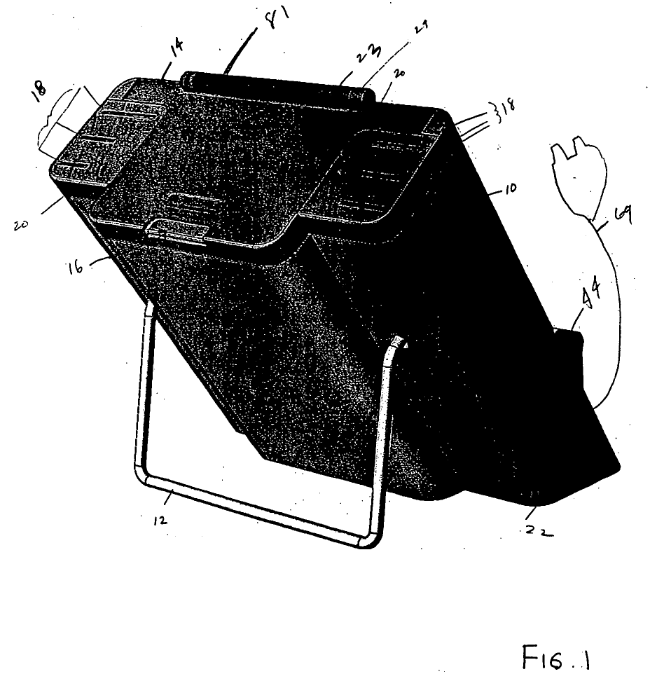

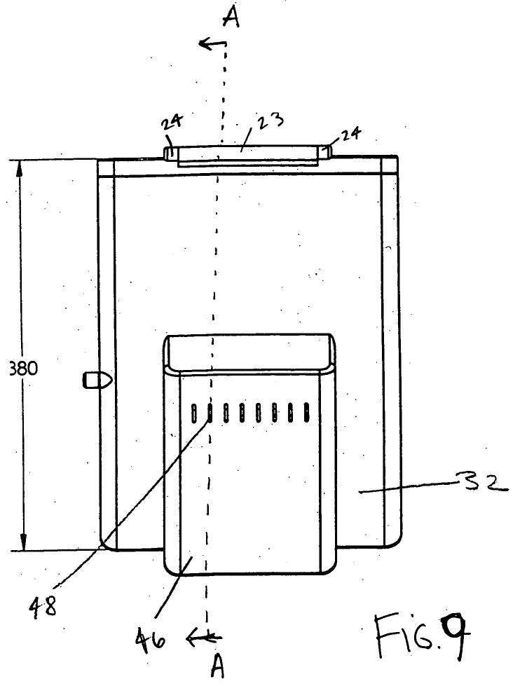

FIG. 1 is a front view of the sterilizer 10 in a closed position. Support 12 enables sterilizer 10 to stand in a semi-upright position. As shown in FIG. 8 in a preferred embodiment, bottom 22 of sterilizer 10 is angled to enable sterilizer 10 to stand and tilt at an angle which is convenient for use. In one embodiment, the plane created by cover 14 in a closed position is maintained at an angle of 60 degrees from the surface on which sterilizer 10 is placed, for ease of use. Cover 14 enables the unit to be closed when it is activated and in a sterilization cycle or when items therein are being stored. UV activation switch 16 activates the UV-C light source located within the sterilizer and begins the sterilization cycle when depressed.

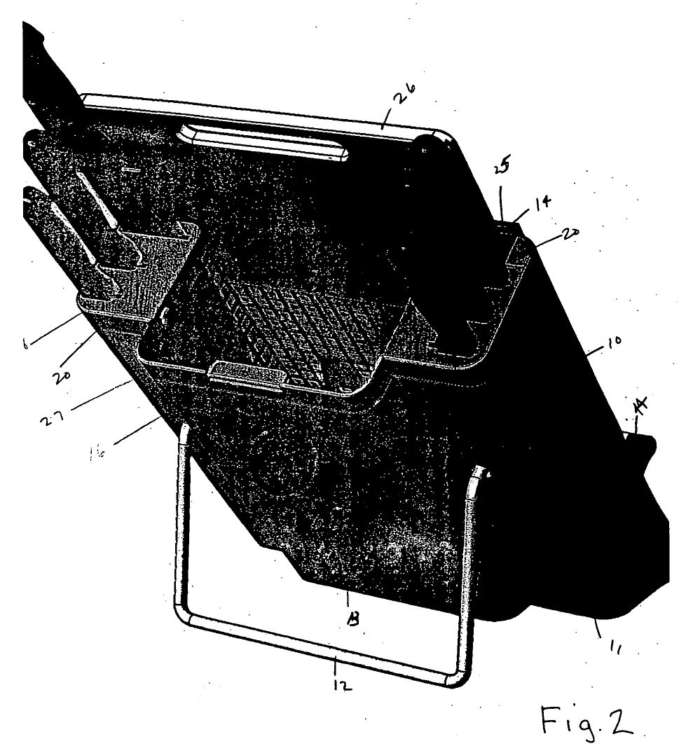

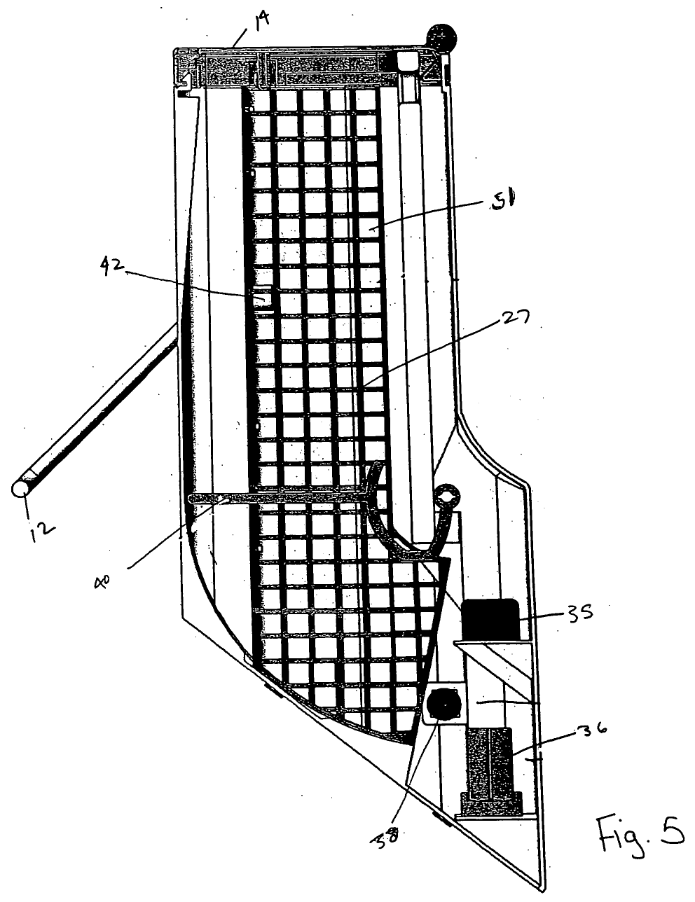

Housing 11 comprises two vertical shells and is made of a material that is impervious to UV-C radiation. In one embodiment housing 11 is formulated from plastic. In another embodiment, as shown in FIG. 2, housing 11 is substantially rectangular in shape, with a rectangular protrusion 13 in the front of housing 11 to accommodate tool holder 27 (partially visible). Knife slots 18 are located in knife holders 20 and enable knives to be stored and sterilized in sterilizer 10. In one embodiment, knife slots 18 are angled such that each knife is maintained at about a 40 degree angle from the horizontal plane created by the surface on which the sterilizer is placed to maximize exposure to UV light within sterilizer 10. Knife holders 20 are fabricated from rigid material, such as plastic with a wood or plastic veneer to maintain the required angle. As shown in FIG. 6, cover 14 is shaped to close over sterilizer 10 in a manner which does not interfere with access to knife holders 20. Hinge 23 of cover 14 enables cover 14 to open and rotate about 270 degrees back behind sterilizer 10 in an open position. As seen in FIG. 2, board slot 25 accommodates cutting board 26 for sterilization and storage. In one embodiment, shown in FIG. 2, tool holder 27 is positioned inside a recess defined by board slot 25, knife holders 20 and protrusion 13 of housing 11.

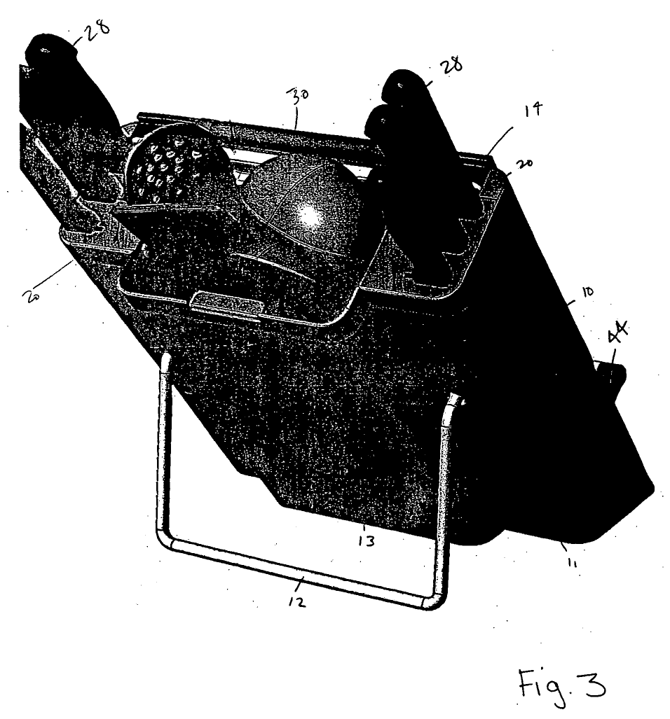

As seen in FIG. 3, in one embodiment, sterilizer 10 accommodates knives 28 and tools 30 as well as cutting board 26 (not visible) for sterilization or storage.

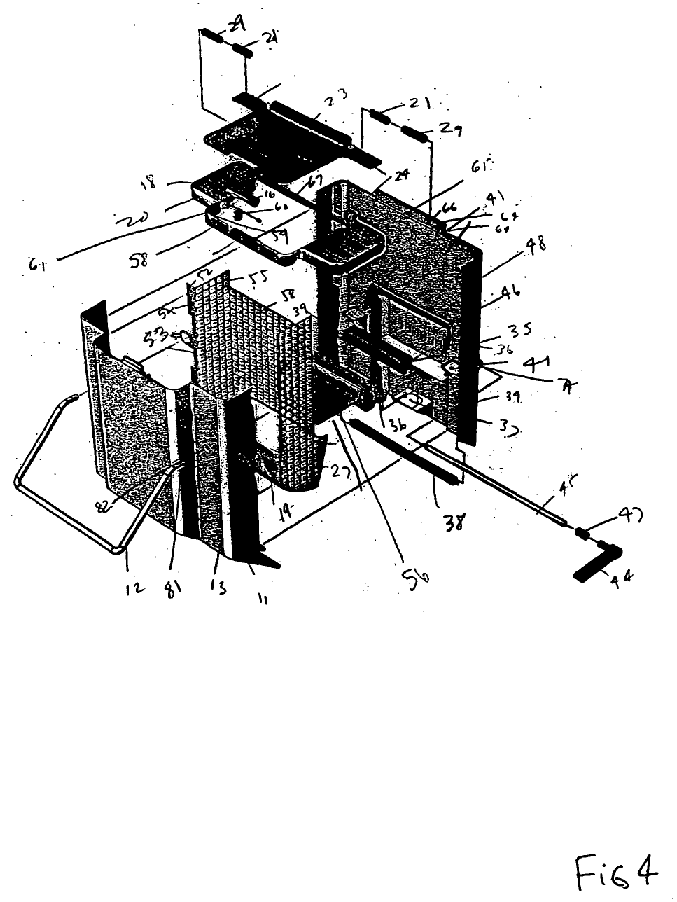

As shown in FIG. 4, which is an exploded view of sterilizer 10 showing the main components, housing 11 comprises front shell 31 and back shell 32. Back shell 32 contains back protrusion 46 which houses electrical ballast 35, starter 36, sockets 39, reflectors 41 and UV-C light source 38. Back shell 32 also contains vents 48 to prevent electrical components housed in back protrusion 46 from becoming overheated. Front shell 31 comprises holes 50 (one not visible) which receive the arms of support 12. Tool holder 27 comprises apertures 51 which enable UV-C light to pass through tool holder 27 and contact items therein. Tool holder 27 comprises three partial holder walls 19. In one embodiment, apertures 51 are symmetrically spaced throughout walls 19 and are of uniform size.

Tool holder 27 is made from material which is durable and not easily cut by sharp tools. In one embodiment, tool holder 27 is made of plastic mesh. The sides of tool holder 27 comprise ridges 52 on either side, each of which further comprises attachment tab 53 and screw hole 54. Tool lever rod 45 is attached to tool cage lever 44 via fastener 47. Tool floor 55 comprises a flat plate 56 rigidly fastened using standard techniques, such as welding, to a semicircular hinge 57. In one embodiment, flat plate 56 and semi-circular hinge 57 are made of an integral piece of molded metal or plastic. Knife holders 20 are seated into collar 58. In a preferred embodiment, collar 58 is fabricated from metal or plastic. In another embodiment, collar 58 contains a rubber sealing ring (not visible) around its inside rim 59 to provide a secure seal when sterilizer 10 is closed and to prevent leakage of UV-C rays.

In assembly, as shown in FIG. 1, cover 14 is movably fitted into collar 58 and secured by a fastener, such as a clip, to form cover assembly 81 as shown in FIG. 6. As shown in FIG. 4, springs 21 are loaded into hinge 23. Hinge caps 29 are then placed onto hinge 23 such that springs 21 exert outward tension on hinge caps 29 when the outward edges of hinge caps 29 are flush with the outside edges of hinge 23. Hinge 23, located on cover 14 is then seated between hinge caps 29 on rim 59. The tension created by springs 21 inside hinge 23 movably secures cover 14 to rim 59 such that cover 14 can move in an arc of about 270 degrees around hinge 23. While the arc may be decreased, an arc of about 270 degrees allows for ease of filling and emptying of tool holder 27 and removal and replacement of cutting board 26. Activator 60 is electrically connected to activation switch 16 and is fastened into notch 61 on rim 59 via fastener 62. Activation switch 16 is seated into notch 63 and rigidly attached thereto using standard techniques, such as soldering. Ballast 35 is fastened to ballast holder 36 using fasteners such as screws. Starter 36 is seated into starter holder 37 and fastened thereto using standard fasteners such as screws. UV-C light source 38 is fastened into sockets 39.

In a preferred embodiment, UV-C light source 38 is cylindrical type G6T5, has a wattage of 6, a base diameter of 15 mm, lamp diameter of 15 mm, is 9 inches in length, has a two 2-pin ceramic bases and is constructed of hard glass quartz. Effective sources of UV-C light are low pressure mercury discharge lamps. A preferred lamp is manufactured by Osram Sylvania, Inc. Another is manufactured by Royal Philips Electronics. Intensity at 1 meter using a type G6T5 bulb 16.7 uW/cm² and 173 uW/cm² In a preferred embodiment, starting voltage of UV-C lamp 50 is 120 VAC @ 60 HZ, operational voltage is 120 VAC @ 60 HZ, UV output is 253.7 nm @ 100 hrs. The average life of a G6T5-type lamp is 6,000 hours.

Activator 60 is electrically connected to starter 36. When pressed, activator 60 transmits an electrical signal to starter 36 which in turn activates UV-C light source 38. UV-C light source 38 is electrically attached to ballast 35, which provides resistance to stabilize current in the circuit created when sterilizer 10 is attached to a power source via power cord 69. In a preferred embodiment, ballast 35 is operational with 100/200 VAC at 50/60 HZ.

Tool holder 27 is attached to front shell 31 by standard fasteners, such as screws, which are placed through attachment tab 53 and screwed into holes 72 on the inside sides of front shell 31. Tool lever rod 45 is placed through hole 74 of back shell 32 and through hole 76 in rod 78 of tool lifter 80, such that a portion of tool lever rod 45 protrudes from the outside of back shell 32. Tool cage lever is attached to the protruding portion of tool lever rod 45 via fastener 42. Screw ends 81 of support 12 are placed through front shell 31 and secured by screw 42, as shown in FIG. 5.

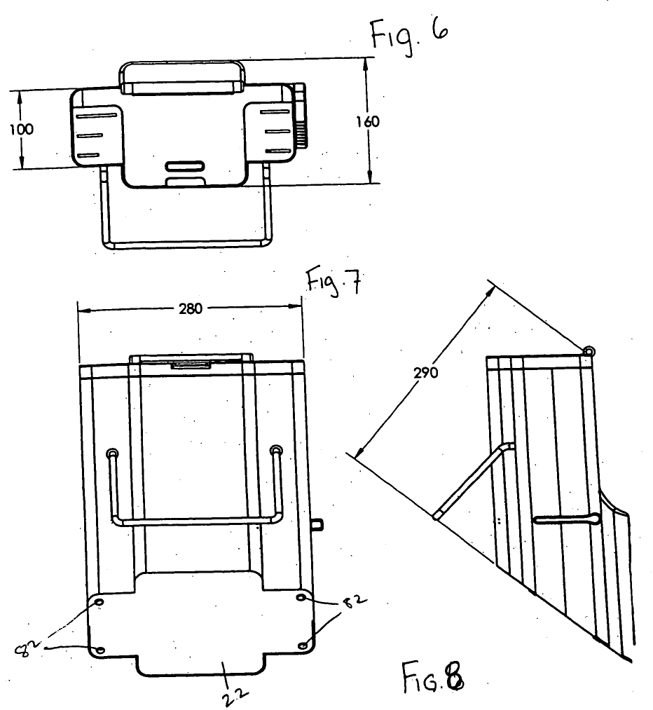

Front shell 31 is then engaged with back shell 32 in a vertical plane such that back shell 32 and front shell 31 form housing 11 and enclose tool holder 27. Front shell 31 and back shell 32 are fastened together using standard fasteners, such as screws. As seen in FIG. 7, bottom 22 is fastened to the bottom edge of back shell 32 and front shell 31 using bottom fasteners 82. In one embodiment, bottom 22 may be covered in a fabric, such as felt, to prevent bottom 22 from scratching surfaces on which sterilizer 10 is placed.

As seen in FIG. 4, cover assembly 81 is placed over and into tab 65 such that bar 67 on rim 59 engages lips 64 (partially visible) on tab 65. Rim 59 can also be rigidly attached to tab 65 using standard fasteners, such as screws.

All internal surfaces of back shell 32, front shell 31, tool floor 55 and cover assembly 81 are coated with UV-C material by vacuum coating or electro-coating or are made of UV-C reflective material, such as polished aluminum or stainless steel, to increase UV-C reflectivity when sterilizer 10 is closed and undergoing a sterilization cycle.

In operation, sterilizer 10 is positioned to be resting on support 12 as seen in FIG. 1, such that cover 14 can be easily accessed. Cover 14 is opened and rotated about 270 degrees to rest on the back of back shell 32, as seen in FIG. 3. As further seen in FIG. 3, knives 28 are placed into knife slots 18 in knife holders 20 such that the blades of the knives 28 are positioned on the sides of tool holder 27 and maintained at about a 40 degree angle from the horizontal plane created by the surface on which sterilizer 10 is placed. For ease of loading, tool lever 44 is rotated such that tool floor 55 is in a raised position and substantially perpendicular to front shell 31 and back shell 32. Kitchen tools 30 are arranged in tool holder 27 such that they rest against tool floor 55. As seen in FIG. 2, cutting board 26 is lowered into board slot 25. Cutting board 26 is pushed down into board slot 25 such that the topmost edge of cutting board 26 is substantially flush with the top edge of back shell 31. Tool lever 44 is then rotated such that tool floor 55 lowers, in turn lowering tools 30 into sterilizer 10 such that the tops of the tools 30 are substantially flush with the top edge of back shell 31. Cover 14 is rotated forward toward front shell 31 and pressed down onto front shell 31 to form a seal, as shown in FIG. 1.

Sterilizer 10 may be utilized in this manner as a storage unit for knives 28, tools 30 and cutting board 26. Such storage maintains these items in a clean, safe and convenient manner.

If it is desired to sterilize such items, cover 14 is closed fully then the activation switch 16 is depressed, completing the electrical circuit inside sterilizer 10 and causing UV-C light source 38 to be activated. UV-C light source 38 emits UV-C radiation, which is directed through apertures 51 and onto the surfaces of knives 28, tools 30 and cutting board 26. UV-C emissions also are reflected off the coated surfaces of back shell 32, front shell 31, tool floor 55 and cover assembly 81 randomly onto the surfaces of knives 28, tools 30 and cutting board 26. As the organic, or carbon based microorganisms on the surfaces of such items are exposed to the UV-C light, the molecular bonds in such microorganisms are broken, causing genetic damage and preventing such organisms from reproducing, rendering them harmless. The ability of UV-C light to disable such microorganisms in this manner is directly related to intensity of UV-C light and exposure time. In a preferred embodiment, activation switch 16 is attached to a timer which enables the sterilizing cycle to proceed for a pre-set period of time. After the time period ends, UV-C light source 38 ceases emitting light. A sterilizing cycle of fifteen minutes has been found to be sufficient to insure sterilizing of six tools, a cutting board and six knifes. After sterilization, cover 14 is opened and rotated behind back shell 32. Lever 44 is rotated such that tool floor 55 is in a raised position and substantially perpendicular to front shell 31 and back shell 32. Kitchen tools 30 are raised up and partially out of sterilizer 10 for ease of retrieval.

What has been illustrated and described herein is a knife and kitchen tool sterilizer and holder. While the invention has been illustrated and described with reference to certain preferred embodiments, the present invention is not limited thereto. In particular, the foregoing specification and embodiments are intended to be illustrative and are not to be taken as limiting. Thus, alternatives, such as structural or mechanical equivalents and modifications will become apparent to those skilled in the art upon reading the foregoing description. Accordingly, such alternatives, changes, and modifications are to be considered as forming a part of the present invention insofar as they fall within the spirit and scope of the appended claims.

1. A sterilizer comprising:
a housing forming an enclosure:
a receptacle in said enclosure;
means for closing said enclosure;
means for holding utensils within said receptacle adjacent said closing means such that said utensils are exposed within said enclosure; and
a source of UV-C radiation located within said enclosure;
wherein UV-C radiation from said source of UV-C radiation is directed onto items located in said enclosure.

2. The sterilizer of claim 1 wherein said means for holding utensils is a knife holder.

3. The sterilizer of claim 1 wherein said receptacle is a basket which has openings of uniform size.

4. The sterilizer of claim 1 wherein said receptacle is of transparent plastic.

5. The sterilizer of claim 1 wherein said means for holding utensils is a receptacle for a cutting board.

6. The sterilizer of claim 1 where the bottom of said housing is angled with respect to said closing means.

7. The sterilizer of claim 1 further comprising means for lifting and lowering utensils located in said receptacle.

8. The sterilizer of claim 1 wherein said UV-C light source emits radiation having a bandwidth between 200 nm and 290 nm.

9. The sterilizer of claim 1 wherein said UV-C light source emits radiation having a bandwidth between 250 nm and 260 nm.

10. The sterilizer of claim 1 further comprising a power switch operated by a timer.

11. The sterilizer of claim 1 wherein items placed inside said enclosure are exposed to UV-C radiation and thereby sterilized.

12. A sterilizer comprising:
An enclosure which can be opened and closed on at least one side;
a receptacle arranged inside said enclosure wherein said receptacle does not interfere with the transmission of the UV-C radiation;
a source of UV-C radiation located within said enclosure;
a power source;
a power switch electrically connected to said source of UV-C radiation and to said power source;
and means for lifting and lowering items placed in said receptacle inside said enclosure, wherein;
when said sterilizer is connected to said power source and said power switch is activated, UV-C radiation from said source of UV-C radiation is uniformly directed onto items located in said receptacle.

13. The sterilizer of claim 12 wherein exposure time of said items to said UV-C radiation is fifteen minutes.
